(19) United States
(12) Patent Application Publication
(10) Pub. No.: US 2010/0010267 A1
(43) Pub. Date: Jan. 14, 2010

(54) ADDITION OF H2S TO TERPENES FOR PRODUCING NOVEL MOLAR MASS REGULATORS FOR RADICAL POLYMERISATIONS

(75) Inventors: Patrick Deck, Mannheim (DE); Oliver Birkert, Fussgonnheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/445,979

(22) PCT Filed: Oct. 15, 2007
(86) PCT No.: PCT/EP2007/060936

§ 371 Date: Apr. 17, 2009

(30) Foreign Application Priority Data
Oct. 17, 2006 (EP) 06122461.4

(51) Int. Cl. C07C 319/04

(52) U.S. Cl. 568/67; 568/61

(57) ABSTRACT

The present invention relates to a method for producing thiols through addition of hydrogen sulfide onto at least one unsaturated terpene, where the reaction is carried out in the presence of at least one organic ion exchanger, and to the use of thiols derived from terpinolene as molar mass regulator in the polymerization of monomers.

The present invention relates to a method for producing thiols through addition of hydrogen sulfide onto unsaturated terpenes, where this reaction is carried out in the presence of at least one ion exchanger as catalyst.

Methods for the addition of hydrogen sulfide onto unsaturated compounds are already known from the prior art.

U.S. Pat. No. 4,140,604 discloses a method for producing mercaptans by reacting ethylenically unsaturated organic compounds with hydrogen sulfide in the presence of ultraviolet radiation. As suitable ethylenically unsaturated compounds, U.S. Pat. No. 4,140,604 discloses straight-chain and branched olefins and cyclic compounds, such as, for example, cyclohexene, 1-methylcyclohexene, vinylcyclohexane, styrene, d-limonene, α-pinene, β-pinene, camphene, p-menthene, α-terpineol and other terpenes.

U.S. Pat. No. 2,076,875 discloses a method for producing terpene mercaptans and sulfides by reacting unsaturated terpenes with hydrogen sulfide in the presence of catalysts comprising acids such as sulfuric acid, phosphoric acid, acetic acid and others or their anhydrides, and bases such as, for example, potassium hydroxide, calcium hydroxide, monoamylamine, diamylamine, triamylamine, metallic aluminum and metal sulfides. It is also possible to use mixtures of catalysts which, besides the abovementioned catalysts, comprise activated charcoal or silica gel.

Flavour and Fragrance Journal, Volume 8, 289 to 294 (1993) discloses a method for producing fragrances through addition of hydrogen sulfide onto monoterpenes in the presence of aluminum trichloride or aluminum tribromide as catalyst.

U.S. Pat. No. 3,408,403 discloses a method for producing mixtures of sulfides and thiols by reacting compounds selected from pinene, dipentene, terpinene, terpinolene, terpineol, α-pinene, β-pinene etc. with hydrogen sulfide in the presence of a catalyst selected from the group consisting of aluminum oxide, silicon oxide-aluminum oxide, bauxite, montmorillonite clay and the like.

DE 28 37 509 discloses a method for producing 1,7,7-trimethylolbicyclo[2,2,1]heptane-2-thiol by reacting α-pinene with gaseous hydrogen sulfide in the presence of a complex compound of boron trifluoride with diethyl ether of the formula BF₃*(C₂H₅)₂O.

The methods for producing thiols through addition of hydrogen sulfide onto unsaturated organic compounds according to the prior art involve the use of acidic or basic organic and inorganic compounds as catalysts. These can on the one hand be dissolved homogeneously in the reaction solution, on the other hand the catalysts can also be present in heterogeneous form. The prior art does not describe a method which involves the use of acidic ion-exchanger resins as catalysts for the addition of hydrogen sulfide onto unsaturated terpenes.

It is an object of the present invention to provide a method for producing thiols through addition of hydrogen sulfide onto unsaturated terpenes which is distinguished by high conversions per used amount of catalyst and problem-free work-up of the reaction mixture.

This object is achieved by a method for producing thiols through addition of hydrogen sulfide onto at least one unsaturated terpene, where the reaction is carried out in the presence of at least one organic ion exchanger.

The method according to the invention is distinguished by the fact that the use of organic ion exchangers considerably simplifies the work-up of the reaction mixture. When the reaction is complete, the catalyst can be separated off from the reaction mixture by simple steps, for example filtration or decantation. Complex steps for separating off a homogeneously dissolved catalyst are not required. Furthermore, acidic organic ion exchangers are characterized by a high density of reactive centers, meaning that the rate of the addition of the hydrogen sulfide onto unsaturated terpenes is considerably increased even in the presence of small amounts of ion exchanger. The ion exchangers have a relatively low density, meaning that they have many active centers, for example per kilogram. A further advantage of ion exchangers is that they can be regenerated in a simple way, for example by treatment with acids. The acidic ion exchangers used have about the same acid strength as sulfuric acid, meaning that they catalyze the reaction according to the invention as well as sulfuric acid, but have the advantages of a solid heterogeneous catalyst (see above).

For producing thiols according to the method of the invention, the at least one unsaturated terpene and hydrogen sulfide are brought into contact in the presence of at least one organic ion-exchanger resin.

The temperature during the reaction of the at least one unsaturated terpene with hydrogen sulfide is generally 20 to 150° C., preferably 70 to 120° C.

The pressure prevailing during the method according to the invention is generally set to 0.5 to 50 bar, preferably 2 to 45 bar, particularly preferably 7 to 35 bar, for example 10 or 30 bar, very particularly preferably 8 to 12 bar. In most cases, it is advantageous to use hydrogen sulfide in excess. A highly suitable ratio of hydrogen sulfide to unsaturated terpene is generally at least 1, preferably at least 1.2 and particularly preferably at least 1.3, and in general at most 20, preferably at most 10 and particularly preferably at most 6 mol of hydrogen sulfide per mole of terpene. For example, a ratio of 1.5 is set. The reaction can be carried out continuously or discontinuously (batchwise). In the case of a discontinuous procedure, the reaction time, and in the case of a continuous procedure, the residence time in the reactor, is adjusted so that the desired conversion is achieved.

Any organic ion exchanger can be used as catalyst in the method according to the invention. In one embodiment, the organic ion exchanger used is an acidic organic ion exchanger.

Organic ion exchangers are solids, for example in particle form, with a three-dimensional, water-insoluble, highly molecular structure, the so-called matrix, to which numerous so-called anchor groups, for example —SO₃⁻, phenolic hydroxy groups or —COO⁻ are covalently bonded. Their loosely bonded counterions, for example Na⁺, H⁺ etc., can be exchanged for other ions of equal charge dissolved in the surrounding liquid. This process is reversible, i.e. during the regeneration of the ion exchanger, the original state is regained. In the method according to the invention, the acidic ion exchangers act as acids by releasing protons.

Examples of acidic ion-exchanger resins are, for example, sulfonated polymers, for example sulfonated styrene or styrene-divinylbenzene polymers. It is also possible to use ion exchangers based on styrene-acrylic acid copolymers which are sulfonated. Organic ion-exchanger resins are either suspended in the reaction mixture or arranged in the form of customary ion-exchanger particles as fixed bed. In one preferred embodiment, the catalyst used is sulfonated styrene, styrene-divinylbenzene polymers and styrene-acrylic acid copolymers, for example Amberlyst 15®.

If the catalyst, for example in the case of a discontinuous procedure, is used in suspended form, then it is generally used in an amount of from 0.1 to 50% by weight, preferably 1 to 20% by weight, particularly preferably in an amount of from 3 to 6% by weight, for example 4.5% by weight, in each case based on the terpene used.

In the method according to the invention, unsaturated terpenes are reacted with hydrogen sulfide to give the corresponding thiols. Within the scope of the present invention, the term “unsaturated” means that the suitable terpenes have at least one double bond in the molecule.

According to the invention, in one preferred embodiment, it is possible to use unsaturated mono-, sesqui-, di- and triterpenes which have at least one double bond in the molecule. Monoterpenes are terpenes which have 10 carbon atoms, sesquiterpenes have 15 carbon atoms, diterpenes have 20 carbon atoms, and triterpenes have 30 carbon atoms.

Suitable unsaturated monoterpenes are, for example, selected from the group consisting of myrcene, (Z)-ocimene, (E)-ocimene, cosmene, linalool, geraniol, nerol, citronellol, myrcenol, geranial, neral, citronellal, geranic acid, α-damascone, β-damascone, β-damascenone, α-terpinene, γ-terpinene, limonene, terpinolene, α-phellandrene, β-phellandrene, 1,3,8-menthatriene, 1-methyl-4-isopropylbenzene, α-terpineol, piperitol, pulegol, dihydrocarveol, carveol, (+)-citronellol, isopulegol, α-thujene, sabinene, thujone, Δ³-carene, α-pinene, β-pinene, camphene, verbenol, verbenone, pinocarveol, myrtenol, myrtenal, β-fenchene, α-fenchene, δ-fenchene and mixtures thereof.

Suitable unsaturated sesquiterpenes are, for example, selected from the group consisting of (E)-α-farnesene, (Z)-α-farnesene, (E)-β-farnesene, α-bisabolene, β-bisabolene, α-bisabolol, zingiberene, α-curcumene, β-curcumene, lanceol, α-elemene, β-elemene, elemol, α-humulene, zerumbone, β-cadinene, γ-cadinene, α-cadinol, δ-cadinol, T-muurolol, T-cardinol, khusol, β-selinene, α-eudesmol, β-eudesmol, rishitin, chrysanthemol, eremophilene, nootkatene, (+)-nootkatene, (+)-valencene, (+)-α-vetivone, guaiol, carotol, velleral and mixtures thereof.

Suitable unsaturated diterpenes are, for example, selected from the group consisting of vitamin A, cembrene A, (−)-sclareol, forskolin, manool, scoparic acid A and mixtures thereof.

Suitable unsaturated triterpenes are, for example, selected from the group consisting of unsaturated steroid hormones, steroid alkaloids, D-vitamins, bile acids, squalene, lanosterol, cycloartenol, agnosterol, 24,25-dihydroanosterol, 24,25-dihydroagnosterol, α-amyrin, βamyrin, ursolic acid, bauerenol, glycyrrhetic acid, oleanolic acid and mixtures thereof.

An unsaturated terpene is very particularly preferably selected from the group consisting of terpinolene, limonene, α-pinene, β-pinene, camphene, p-menthene and mixtures thereof, particularly preferably terpinolene or limonene.

Thus, in a particularly preferred embodiment, the present invention comprises the conversion of terpinolene (1) to the corresponding thiols,

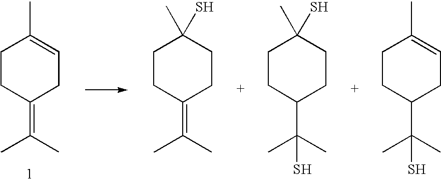

The thiols derived from the terpenes that are very particularly preferred according to the invention, preferably terpinolene or limonene, are distinguished by particularly pleasant odors, for example the thiols derived from terpinolene smell of grapefruit. Furthermore, said thiols are nontoxic.

The terpenes which can be used according to the invention can be prepared by the method known to the person skilled in the art, see, for example, Römpp, Chemielexikon, 9th edition, pages 4508 to 4510, or are commercially available. A further method of obtaining terpenes is biosynthesis with subsequent isolation by methods known to the person skilled in the art.

The method according to the invention can, if desired, be carried out without solvents or in the presence of a solvent. If a solvent is used, then use is made of a solvent which is inert toward the reactants. Examples of inert solvents are hydrocarbons or hydrocarbon mixtures, such as pentane, hexane, cyclohexane, benzine, benzene, toluene or xylene. However, the reaction is preferably carried out in the absence of a solvent.

The reaction of hydrogen sulfide with the at least one unsaturated terpene is carried out continuously in one embodiment. In a further embodiment, the reaction of hydrogen sulfide with the at least one unsaturated terpene is carried out discontinuously. In general, the reaction can be carried out in any suitable reaction apparatus, for example stirred-tank reactors in the case of a discontinuous procedure, tubular reactors in the case of a continuous procedure, with a suspended or fixedly arranged catalyst.

In a preferred embodiment, the at least one terpene to be reacted is presaturated with H₂S in a separate reactor under pressure and then pumped into the reactor filled with catalyst. It is a further preferred embodiment to initially introduce the catalyst and the terpene into the reactor, and then to inject hydrogen sulfide at the above-stated pressure.

To work up the thiol produced, the reactor product is, if necessary, freed from the solvent. The catalyst present, if appropriate, in the crude product is separated off by suitable methods known to the person skilled in the art, for example filtration or decantation. Usually, the thiol produced is purified using customary methods; this can take place, for example, by fractional crystallization at low temperature or by distillation, preferably under reduced pressure.

The thiols according to the invention are highly suitable as molar mass regulators in the polymerization, preferably the free-radical polymerization, of monomers.

Methods for the free-radical polymerization of vinylic monomers using thiols produced by the method according to the invention as molar mass regulators are known. Examples of vinylic monomers which are converted to polymers in a known manner by free-radical polymerization, where the thiols produced according to the invention are used as molar mass regulators, are butadiene, styrene, carboxylated styrene, acrylic acid, acrylonitrile, acrylic ester, vinyl ether or their mixtures. These monomers are often polymerized in the form of an emulsion in a solvent, for example and preferably water, in which case a dispersion of the polymer in water is formed.

The thiols according to the invention, particularly the thiols derived from terpinolene, are suitable, on account of their good odor and their nontoxic nature, particularly as molar mass regulators in the polymerization of monomers. The present invention therefore also relates to the use of thiols derived from terpinolene as molar mass regulator in the polymerization, preferably the free-radical polymerization, of monomers.

### EXAMPLES

### Example 1

Terpinolene is reacted at a temperature of 100° C. and a pressure of 10 bar with hydrogen sulfide for 8 hours in the presence of Amberlyst 15, an acidic ion exchanger, to give a mixture of the mono- and dithiol comprising the corresponding possible isomers. The desired isomer is separated off by distillative separation.

### Example 2

100.0 g (0.73 mol) of limonene (purum, Fluka) are reacted in the presence of 4.5 g of Amberlyst 15, which has been dried beforehand at room temperature under reduced pressure, in a 0.3 1 autoclave with gas-dispersion stirrer stirred from above with magnetic stirrer coupling, continuous stirrer shaft, thermocouple, heating bath (electric)) with H₂S (from 0.500 kg steel cylinder, Messer-Griesheim) at a pressure of 9.9 bar and a temperature of 100° C. for a period of eight hours. For this, limonene and catalyst are initially introduced into the autoclave, and the autoclave is sealed and injected with nitrogen. The stirrer is turned up to a speed of 500 rpm, and the autoclave is flushed three times with 10 bar of H₂S, decompressed and heated to 100° C. At a stirrer speed of 800 rpm, H₂S is injected at 10 bar. After a reaction time of eight hours, the autoclave is decompressed, the reactor contents are flushed three times with about 10 bar of N₂, the catalyst is separated off via a filter, and the crude product is purified by distillation. This gives 76 g of a mercaptan mixture, which corresponds to a yield of 61%, and a selectivity of 76%.

1-8. (canceled)

9. A method for producing thiols which comprises adding hydrogen sulfide onto at least one unsaturated terpene in the presence of at least one organic ion exchanger.

10. The method according to claim 9, wherein the at least one unsaturated terpene is selected from the group consisting of terpinolene, limonene, α-pinene, β-pinene, camphene, p-menthene and mixtures thereof.

11. The method according to claim 9, wherein the at least one organic ion exchanger is selected from the group consisting of sulfonated styrene, styrene-divinylbenzene polymer and styrene-acrylic acid copolymer.

12. The method according to claim 9, wherein the method is carried out at in a reaction at a temperature of from 20 to 150° C.

13. The method according to claim 9, wherein the method is carried out at in a reaction in the absence of a solvent.

14. The method according to claim 9, wherein the method is carried out at in a continuous reaction.

15. The method according to claim 9, wherein the method is carried out at in a batch reaction.

16. The method according to claim 10, wherein the at least one organic ion exchanger is selected from the group consisting of sulfonated styrene, styrene-divinylbenzene polymer and styrene-acrylic acid copolymer.

17. The method according to claim 16, wherein the method is carried out at in a reaction at a temperature of from 20 to 150° C. and a pressure of 0.5 to 50 bar.

18. The method according to claim 17, wherein the method is carried out at in a reaction in the absence of a solvent.

19. The method according to claim 18, wherein the method is carried out at in a continuous reaction.

20. The method according to claim 18, wherein the method is carried out at in a batch reaction.

21. The method of using thiols derived from terpinolene as molar mass regulator in the polymerization of monomers.

22. The method according to claim 9, wherein the method is carried out at in a reaction at a temperature of from 70 to 120° C. and a pressure of 2 to 45 bar.

23. The method according to claim 18, wherein the method is carried out at in a reaction at a temperature of from 70 to 120° C. and a pressure of 8 to 15 bar.

24. The method according to claim 9, wherein the ratio of hydrogen sulfide to unsaturated terpene is at least 1.

25. The method according to claim 9, wherein the ratio of hydrogen sulfide to unsaturated terpene is 1.2 to 20.

26. The method according to claim 9, wherein the ratio of hydrogen sulfide to unsaturated terpene is 1.3 to 6.

27. The method according to claim 18, wherein the ratio of hydrogen sulfide to unsaturated terpene is 1.3 to 6.
